(19) United States
(12) Patent Application Publication
(10) Pub. No.: US 2019/0216445 A1
(43) Pub. Date: Jul. 18, 2019

(54) INSTRUMENTED BIOPSY PROBE

(71) Applicants: Thomas Ried, (US); THE CHARLES STARK DRAPER LABORATORY, INC., Cambridge, MA (US)

(72) Inventors: Andrew A. Berlin, Lexington, MA (US); Salil Desai, Cambridge, MA (US); Thomas Ried, Bethesda, MD (US); Chris Salthouse, Newton, MA (US)

(21) Appl. No.: 16/368,318

(22) Filed: Mar. 28, 2019

Related U.S. Application Data
(62) Division of application No. 15/683,381, filed on Aug. 22, 2017.
(60) Provisional application No. 62/378,043, filed on Aug. 22, 2016.

(51) Int. Cl. A61B 10/02; A61B 5/00; A61B 5/053

(52) U.S. Cl. CPC A61B 10/0233; A61B 5/0075; A61B 10/0041; A61B 10/0283; A61B 5/0538; A61B 5/6848

(57) ABSTRACT

A system for collecting a sample from a tissue includes a medical instrument having a sample collection region. One or more sensors is configured to detect one or more properties of a sample, and is configured to output at least one measured value representative of the one or more properties of the sample. An indicator is operatively connected to the one or more sensors and is configured to provide a notification to a user of the medical instrument based on the one or more properties of the sample detected by the at least one sensor. A method of guiding sample collection is also provided.

### CROSS REFERENCE TO RELATED APPLICATIONS

This application is a divisional of pending U.S. patent application Ser. No. 15/683,381, titled “Instrumented Biopsy Probe,” filed Aug. 22, 2017, which claims priority to U.S. provisional patent application 62/378,043, filed Aug. 22, 2016, each of which is incorporated by reference herein in its entirety for all purposes.

### FIELD OF THE DISCLOSURE

The present disclosure covers the concept of introducing a sensing mechanism to characterize acquired samples into a surgical instrument used to conduct biopsies.

### SUMMARY

The present disclosure introduces a medical instrument such as an instrumented biopsy probe/needle that characterizes the acquired samples during the biopsy procedure. Characterizing samples as they are acquired by the medical instrument permits the surgeon to know whether the suspicious cells have been acquired in sufficient quantity to enable analysis, and, in some cases, an instantaneous diagnosis. Advantageously, analysis of the acquired sample may be combined with techniques that characterize tissue surrounding the needle, so as to trigger sample acquisition when the needle is in the presence of suspicious tissue, and to compare with properties of the collected sample to determine whether the suspicious material was successfully acquired.

A variety of techniques to characterize acquired samples are presented, including electrical spectroscopy, cell size measurement, cell wall thickness measurement, optical (infrared (IR)) spectroscopy, Raman spectroscopy, elastometry, and acoustic characterization. Methods to prepare the sample for analysis, such as ultrasonification, dilution, and mixing are also presented. Finally, user interface concepts for such a medical instrument are introduced, including on-needle displays, tactile feedback, and wireless linkage with medical imaging equipment such as ultrasound and magnetic resonance imaging (MRI). For small sample sizes, characterizing the number of cells is important. Cells can be counted based on the above measurements. For example, cells can be counted based on cell size measurements.

According to one aspect of the present disclosure, a system for collecting a sample from a tissue includes a medical instrument having a sample collection region; at least one sensor configured to detect at least one property of a sample, the at least one sensor configured to output at least one measured value representative of the at least one property of the sample; an indicator operatively connected to the at least one sensor and configured to provide a notification to a user of the medical instrument based on the at least one property of the sample detected by the at least one sensor.

In some embodiments, the at least one sensor is positioned on a peripheral device.

In some embodiments, the at least one sensor is positioned on the medical instrument and is configured to detect the at least one property of the sample when the sample is collected in the sample collection region.

In some embodiments, the indicator is positioned on a display on the medical instrument.

In some embodiments, the indicator is positioned on a display on a peripheral device.

In some embodiments, a body of the medical instrument is in the form of a biopsy needle.

In some embodiments, the biopsy needle has a distal end and a proximal end, and the sample collection region is located at the distal end.

In some embodiments, the at least one sensor is embedded in a body of the medical instrument.

In some embodiments, the at least one sensor is secured to a surface of the sample collection region.

In some embodiments, the system includes a sample evaluation module configured to compare the at least one measured value to at least one of a known range of values corresponding to a normal condition and a known range of values corresponding to an abnormal condition.

In some embodiments, the sample evaluation module is operatively connected to the at least one sensor.

In some embodiments, the sample evaluation module is operatively connected to the at least one sensor by a wireless connection.

In some embodiments, the system further includes at least one environment sensor secured to the medical instrument and configured to detect at least one property of a tissue.

In some embodiments, the system further includes a sample comparison module configured to compare the at least one measured value to at least one measured environmental value of the tissue.

In some embodiments, the sample comparison module is operatively connected to the at least one sensor.

In some embodiments, the sample comparison module is operatively connected to the at least one sensor by a wireless connection.

In some embodiments, the at least one property of the sample is selected from the group consisting of: a chemical property, a physical property, an electrical property, and combinations thereof.

In some embodiments, the at least one property of the sample is selected from the group consisting of: a color, a density, a pH, an electrical impedance, an optical spectroscopic property, an elasticity, a mass, an average particle size, a cell size, a cell shape, a cell wall thickness, a temperature, a number of cells within the sample, a cell cluster size, and combinations thereof.

In some embodiments, the medical instrument comprises an ultrasonication device.

In some embodiments, the system further includes a sample preparation module.

In some embodiments, the system further includes an automatic trigger that causes the medical instrument to initiate sample collection when the at least one environment sensor detects a suspicious tissue.

In some embodiments, the sample includes a plurality of cells.

In some embodiments, the plurality of cells includes at least one normal cell and at least one abnormal cell.

In some embodiments, the at least one normal cell is non-cancerous and the at least one abnormal cell is cancerous.

According to another aspect of the present disclosure, a method of guiding sample collection includes maneuvering a device in a tissue; collecting a sample from the tissue using the device; identifying a property of the sample; identifying a property of the tissue; determining whether the property of the sample matches the property of the tissue; and outputting a real-time instruction during the step of maneuvering the device to increase sample acquisition aggressivity if the property of the sample does not match the property of the tissue.

In some embodiments, the step of outputting the instruction comprises one of outputting the instruction to a user and outputting the instruction to a controller.

In some embodiments, increasing sample acquisition aggressivity includes at least one of adjusting of vacuum pressure, adjusting ultrasonication, adding a chemical disrupting agent, and agitating.

In some embodiments, the step of determining is performed on one of the device and a peripheral device.

In some embodiments, the method further includes determining whether the collected sample is abnormal.

In some embodiments, the method further includes informing a user of information related to at least one of the property of the sample and the property of the environment.

In some embodiments, the sample includes a plurality of cells.

In some embodiments, the plurality of cells includes at least one normal cell and at least one abnormal cell.

In some embodiments, the at least one normal cell is non-cancerous and the at least one abnormal cell is cancerous.

Another aspect of the present disclosure provides a system for collecting a sample from a surrounding tissue. The system comprises a medical instrument having a sample collection region and at least one sensor configured to detect at least one property of a sample. The at least one sensor is configured to output at least one measured value representative of a property of the sample. An indicator is operatively connected to the at least one sensor and configured to provide a notification to a user of the medical instrument based on the at least one property of the sample detected by the at least one sensor.

In some embodiments, the indicator is positioned on a display on the medical instrument.

In some embodiments, the indicator is positioned on a display on a peripheral device.

In some embodiments, a body of the medical instrument is in the form of a biopsy needle.

In some embodiments, the biopsy needle has a distal end and a proximal end. A tip of the biopsy needle is located at the proximal end. The sample collection region is located at the proximal end and is connected to the tip by a lumen defined within the body.

In some embodiments, the at least one sensor is embedded in a body of the medical instrument.

In some embodiments, the at least one sensor is secured to a surface of the sample collection region.

In some embodiments, the system includes a sample evaluation module configured to compare the at least one measured value to a known range of values corresponding to a normal condition and/or a known range of values corresponding to an abnormal condition. The sample evaluation unit comprises a memory component and a processor connected to the memory component.

In some embodiments, the sample evaluation module is operatively connected to the at least one sensor.

In some embodiments, the sample evaluation module is operatively connected to the at least one sensor by a wireless connection.

In some embodiments, the system includes at least one environment sensor secured to the medical instrument and configured to detect at least one property of a surrounding tissue.

In some embodiments, the system includes a sample comparison module configured to compare the at least one measured value to at least one measured environmental value of the surrounding tissue. The sample comparison module includes a memory component and a processor connected to the memory component.

In some embodiments, the sample comparison module is operatively connected to the at least one sensor.

In some embodiments, the sample comparison module is operatively connected to the at least one sensor by a wireless connection.

In some embodiments, the at least one property of the sample is a color, a density, a pH, an electrical impedance, an optical spectroscopic property, an elasticity, a mass, an average particle size, a cell size, a cell shape, a cell wall thickness, a temperature, a number of cells within the sample, a cell cluster size, and combinations thereof.

In some embodiments, the at least one property of the sample is a chemical property, a physical property, an electrical property, and combinations thereof.

In some embodiments, the medical instrument comprises an ultrasonication device.

In some embodiments, the system includes a sample preparation module.

In some embodiments the system includes an automatic trigger that causes the medical instrument to initiate sample collection when the at least one environment sensor detects a suspicious tissue.

Another aspect of the present disclosure provides a medical instrument for collecting a sample from a surrounding tissue and for communicating with a processor. The medical instrument includes a sample collection region, and at least one sensor configured to detect at least one property of a sample when the sample is collected in the sample collection region. The medical instrument is configured to communicate information related to the at least one property of the sample to a processor.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows a cross-sectional view of an embodiment of an instrumented biopsy probe;

FIG. 2 shows a cross-sectional view of another embodiment of an instrumented biopsy probe;

FIG. 3 is a block diagram for use with the instrumented biopsy probe of FIG. 1 or FIG. 2;

FIG. 4A shows an end view of a filter for a needle tip;

FIG. 4B shows an embodiment of a flow-focusing device;

FIG. 4C shows a detection region of a cell counter;

FIG. 4D shows a collection region;

FIG. 5 shows a cross-sectional view of another embodiment of an instrumented biopsy probe;

FIG. 6 shows a cross-sectional view of another embodiment of an instrumented biopsy probe;

FIG. 7 shows a cross-sectional view of an embodiment of an assembly;

FIG. 8 shows another view thereof;

FIG. 9 is a block diagram of a method of the present disclosure;

FIG. 10A shows a schematic view of a cell counter being used to count cancer cells;

FIG. 10B shows a plot of normalized impedance over time in two zones of the cell counter of FIG. 10A;

FIG. 11A shows a schematic view of a cell counter being used to count cancer cells; and

FIG. 11B shows a plot of normalized impedance over time in two zones of the cell counter of FIG. 11B.

### DETAILED DESCRIPTION

There are many instances in health care in which a biopsy sample must be acquired. Samples may be acquired surgically via “open biopsy,” but it is often preferable to use a minimally-invasive sample acquisition approach, such as fine-needle aspiration or core-needle biopsy. There has been much work done on “smart biopsy needles” that sense the properties of the tissues surrounding them, so as to trigger sample acquisition when the needle is located in the suspicious tissue. The present disclosure introduces the concept of incorporating sensors on board the sample capture instrument to analyze the acquired sample while the biopsy procedure itself is being performed. In some embodiments, sensors are on a peripheral device near the sample capture instrument to analyze the acquired sample during the biopsy procedure.

Approximately half a million thyroid nodule fine needle aspirate biopsies were performed in the United States alone in 2011. Of these, up to 30% (150,000) yield indeterminate results. For biopsies on parts of the body such as lymph nodes and the thyroid gland, one of the challenges is that cystic fluid or other material, rather than a sample of the diseased tissue, will often be acquired. This is referred to as “insufficient cellularity” or “insufficient sample,” which precludes effective pathological analysis. For certain types of biopsies, even experienced surgeons experience unacceptably high rates of “insufficient sample” biopsies. This can cause the patient to have to return to a medical practitioner to undergo the procedure again, or to have to make medical decisions without the benefit of a biopsy result. For instance, current literature reports a rate of insufficient sample for fine-needle aspiration of nonpalpable breast lesions of 33.95%, rendering use of fine-needle aspiration impractical for that lesion type.

The present disclosure provides a device, a system, and a method related to an instrumented biopsy probe approach useful in various medical procedures, such as breast biopsies (especially in the area of the nipple where use of core-needle biopsy is contra-indicated), lymph node biopsies, thyroid biopsies, lung biopsies, bronchial tube biopsies, and biopsies of metastasized tumor sites for which it is difficult to know whether the tumor has been correctly targeted.

For performance of procedures such as fine-needle aspiration, which is an acquired skill, experienced biopsy surgeons can often reduce their rate of “insufficient sample” biopsies over time, dramatically, relative to less experienced surgeons. This learning curve is limited in part by the typically long delay (such as hours or days) from when the procedure is performed to when pathology results become available informing the surgeon of the adequacy of the sample. Shortening the time from sample collection to providing feedback to the surgeon about sample adequacy thus has the potential to shorten the time required to learn to be an experienced surgeon, since the surgeon will have nearly instant feedback with which to improve their technique. Ideally, the time from sample collection to feedback regarding adequacy can be reduced to the point where the surgeon has feedback in near real-time, while the biopsy procedure is still underway, and can alter the surgical technique while the biopsy is still in progress so as to maximize the adequacy of the acquired sample.

According to an aspect of the present disclosure, a medical instrument is configured for collecting a sample from a tissue and for providing real-time information regarding the sample.

The term “sample” as used herein means a tissue portion comprising cells and/or fluid and/or extracellular material. During a biopsy, harvested cells may include one or more types of cells. Harvested cells may include normal cells, abnormal cells. “Abnormal cells” include cancerous cells, pre-cancerous cells, or cells with unexpected characteristics.

The term “operatively connected” means that one or more components are in communication with each other. For example, a signal such as a signal containing sensed data or a command signal can be communicated between components that are operatively connected.

In some embodiments, the sample is a biopsy sample collected from a living patient. In some embodiments, the sample is collected from a tissue that has been removed from a patient. In some embodiments, the real-time information may be provided while the medical instrument is still inserted in the patient. In some embodiments, the real-time information may be provided after the medical instrument has been removed from the patient, but still during a biopsy procedure so a physician can immediately reuse the medical instrument to remove a second or subsequent sample from the patient.

The real-time information may be based on a property detected by at least one acquired sample sensor. A property detected by an acquired sample sensor may include an electrical impedance spectroscopic signature, an electrical or optical counting of cells, a counting of cell clusters, a measurement of cell wall thickness, optical spectroscopic signatures, optical scattering signatures, Raman spectroscopic signatures, elastometry, mass, pH, temperature, a chemical property such as oxygen concentration or chemical biomarkers (or biochemical marker) associated with events such as cell apoptosis or glycolysis, and an acoustic property, or any combination of these. In some embodiments, cell size and shape can be inferred from electrical impedance of the sample.

The at least one acquired sample sensor may be positioned on the medical instrument or on a peripheral device. For example, an acquired sample sensor may be positioned on a medical instrument such as a biopsy needle, a biopsy punch, a scalpel, an excision tool, a probe, or another medical instrument that may encounter tissue. The acquired sample sensor(s) may be positioned within a lumen of a needle, in a syringe body, or in another part of a medical instrument.

Additionally or alternatively, an acquired sample sensor may be positioned on a peripheral device that is configured to receive the sample from the medical instrument. For example, a physician can use a medical instrument to remove a sample from a patient, and then transfer the sample to the peripheral device where an acquired sample sensor detects at least one property of the sample.

In some embodiments, the medical instrument includes a sample collection region and at least one acquired sample sensor configured to detect at least one property of the sample when the sample is collected in the sample collection region.

In some embodiments, the medical instrument is configured to communicate information related to one or more properties to a processor.

In some embodiments, an indicator is operatively connected to the one or more acquired sample sensors and is configured to provide a notification to a user of the medical instrument based on the one or more properties of the sample detected by the one or more acquired sample sensors. For example, the indicator may be on a display positioned on the medical instrument or on a peripheral device.

In an exemplary embodiment, a cell counter that operates based on electrical impedance detection is incorporated into the center channel of a needle that is used for fine-needle aspiration biopsies. The sample characterization sensing mechanism provides the surgeon with real-time feedback or near real-time feedback about the number of cells contained in the acquired samples. Having feedback permits the surgeon to gauge the adequacy of the acquired sample, thereby knowing whether to discontinue the procedure or to alter the surgical technique. For instance, if a sufficient number of cells are not being acquired, the surgeon could decide to activate an on-board ultrasonication device to enhance sample acquisition, could alter the position or angle of attack of the biopsy probe, could adjust sample acquisition procedural parameters such as the amount of vacuum force used to acquire the sample, or could introduce a chemical disrupting agent.

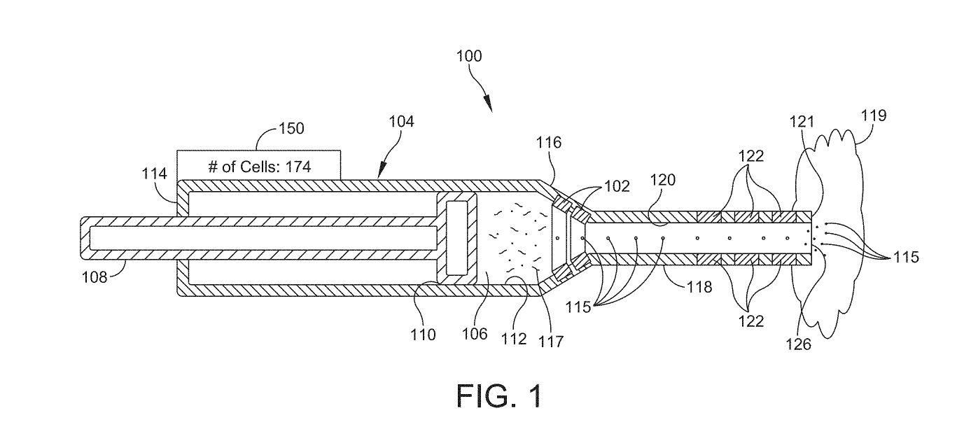

FIG. 1 shows a cross-sectional view of an example of a medical instrument according to the present disclosure. FIG. 1 shows an embodiment of an instrumented biopsy probe 100 with internal acquired sample sensors 102 to detect, quantify, and characterize acquired samples and external sensors to characterize the tissue that is adjacent or nearby the probe where the sample is collected.

The instrumented biopsy probe 100 includes an instrument body 104 that may have a cylindrical body portion 106.

The instrumented biopsy probe includes a negative pressure source. In some embodiments, the negative pressure source may be a pump connected to the instrumented biopsy probe. In some embodiments, the negative pressure source may be a plunger that is movable within the instrumented biopsy probe.

In FIG. 1, a plunger 108 is positioned within the cylindrical body portion 106. An outer edge 110 of the plunger engages an inner surface 112 of the cylindrical body portion 106. The plunger 108 is movable within the cylindrical body portion 106. Movement of the plunger towards the distal end 114 of the cylindrical body portion 106 creates negative pressure within the cylindrical body portion 106.

In FIG. 1, a frustoconical portion 116 extends from the cylindrical body portion 106. A needle 118 having a lumen 120 defined within the needle 118 extends from the frustoconical portion 116 at the proximal end of the syringe body.

A user can operate the instrument body 104 to draw fluid and cells into the instrument body 104. Moving the plunger 108 distally within the cylindrical body portion 106 creates negative pressure within the instrument body 104 to draw a sample 117 into the biopsy needle from a surrounding environment.

FIG. 1 shows the proximal end of the needle 118 positioned adjacent to a tumor 119. The sample 117 may include fluid and cells. If the site being biopsied is truly a tumor 119, and if the biopsy is successful, the sample 117 includes cells 115 from the tumor 119.

In some embodiments, the lumen 120 is configured to allow cells to pass single-file through the lumen 120. This can be accomplished via flow-focusing, via dimensioning the inner diameter of the lumen to be sufficiently narrow, or via a combination of these. In some embodiments, the inner diameter of the lumen is just wide enough to permit a cancer cell to pass through the lumen. In some embodiments, the lumen is as wide as necessary to take in a desired sample. In most instances, the desired sample is cells, such as human cells. In some embodiments, the inner diameter of the lumen 120 of the needle 118 is 200 microns.

The instrumented biopsy probe 100 may be dimensioned to suit the needs of a sample collection procedure.

In some embodiments, the instrumented biopsy probe is a cylindrical tube having a substantially constant outer diameter along its length. In some embodiments, the instrumented biopsy probe is a flexible tube. In some embodiments, the instrumented biopsy probe includes a catheter. In some embodiments, the instrumented biopsy probe includes a camera and/or a light to assist with guidance of the instrumented biopsy probe. In some embodiments, an instrumented biopsy probe according to the present disclosure can be used with other tools to obtain samples.

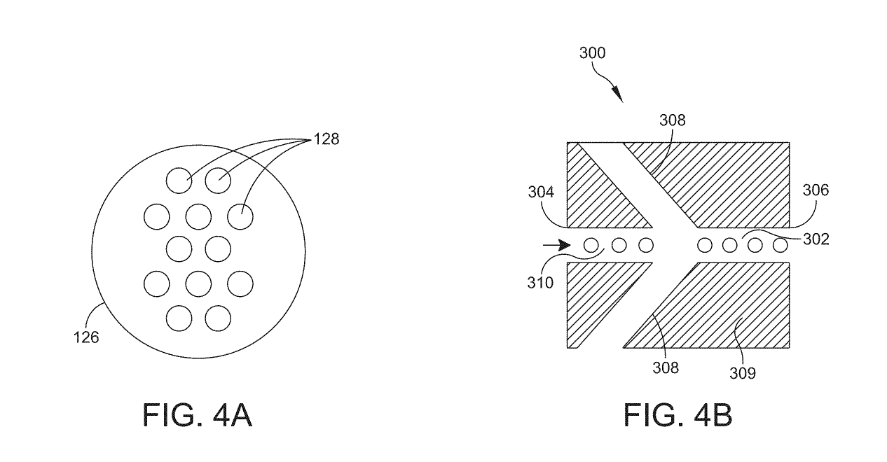

The proximal end of the needle 118 may include a filter 126, such as the filter shown in FIG. 4A. The filter includes pores that are dimensioned to prevent large aggregates of cells or portions of tissue from entering the needle. In some embodiments, the filter includes pores that are just large enough to allow certain individual cells, such as cancer cells and blood cells, to pass through the filter and into the needle.

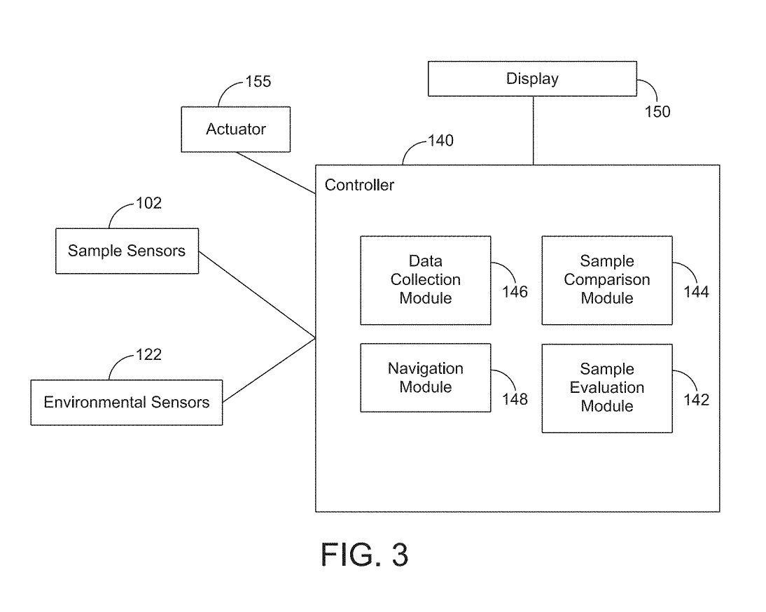

In some embodiments, the proximal end of the needle is actuated. For example, an actuator, such as actuator 155 in FIG. 3, can be used to oscillate the proximal end 121 of the needle 118. This oscillation is useful for breaking up aggregates of cells into individual cells that can pass into the needle through the filter 126.

To determine whether the sample includes cells from the tumor 119, at least one acquired sample sensor 102 is secured to the inner surface of the frustoconical portion 116. FIG. 1 shows two sample sensors 102 secured to the inner surface of the frustoconical portion 116. The acquired sample sensors 102 in FIG. 1 are circular and are concentric with the body of the biopsy probe 100.

In some embodiments, the acquired sample sensors are linear. In some embodiments, the acquired sample sensors are planar. In some embodiments, the acquired sample sensors are another shape that is configured to suit the needs of the sensing application and configured to suit the geometry of the medical instrument on which the acquired sample sensors are secured.

In some embodiments, there are more than two or fewer than two sample sensors secured within the instrumented biopsy probe. In some embodiments, there is one acquired sample sensor secured within the instrumented biopsy probe.

In some embodiments, the acquired sample sensors 102 are secured to the inner surface of the frustoconical portion 116 using an adhesive or another mounting means. In some embodiments, the acquired sample sensors 102 are embedded in the inner surface of the frustoconical portion 116.

The acquired sample sensors 102 are capable of detecting and characterizing properties of the acquired sample 117 during the biopsy procedure. The acquired sample sensors 102 are configured to output at least one measured value representative of the at least one property of the sample to a controller, such as controller 140 of FIG. 3. Properties of the acquired sample 117 may include electrical impedance spectroscopic signature, fluorescence-based light emission, electrical or optical counting of cells, counting of cell clusters, measurement of cell wall thickness, optical spectroscopic signatures, optical scattering signatures, Raman spectroscopic signatures, elastometry, mass, pH, temperature, chemical properties such as oxygen concentration or chemical biomarkers (or biochemical markers) associated with events such as cell apoptosis or glycolysis, and acoustic properties. In some embodiments, cell size and shape can be inferred from electrical impedance of the sample. Where electrical sensors are used, they can be connected to a power supply. When optical sensors are used, they can be connected to an optical waveguide in the medical instrument and a light source.

To determine whether the proximal end 121 of the needle 118 is located adjacent a suspicious tissue, the proximal end of the syringe body includes at least one environment sensor 122. FIG. 1 shows six environment sensors 122 secured to an outer surface of the needle. In some embodiments, each environment sensor is secured to the medical instrument, such as a biopsy probe. Each environment sensor is configured to detect at least one property of a tissue adjacent to the environment sensor.

The acquired sample sensors 102 and the environment sensors 122 are in communication with a controller, which includes a processor. FIG. 3 shows a controller 140 connected to each of the acquired sample sensors 102 and connected to each of the environment sensors 122. The connection between the controller 140 and the acquired sample sensors 102 and the connection between the controller 140 and the environment sensors 122 may be a wired connection or a wireless connection. For example, a wired connection can be a conventional wired connection from the acquired sample sensors 102 to a controller 140 on the medical instrument or on a peripheral device. A wired connection may be a connection such as a radio frequency (RF) connection or an infrared (IR) connection. The controller 140 receives measurements from the acquired sample sensors 102 and measurements from the environment sensors 122. The controller 140 can compare the properties of the acquired sample with those of the tissue, so as to ascertain whether the suspicious tissue (in the case of FIG. 1, tissue from the tumor 119) was acquired. For instance, properties of interest may include at least one of the color of the acquired sample, as well as the density, pH, electrical impedance, optical spectroscopic properties (such as IR and Raman spectroscopic signatures), elasticity, mass, average particle size, average cell aggregate size, size and shape of the acquired cells, number of acquired cells, cell cluster size, chemical properties such as oxygen concentration, and physical properties of the acquired cells, such as cell wall thickness and nuclear size. For example, electrical property measurements may be utilized to characterize the acquired sample. A sensed color may be based on a sensed fluorescence-based light emission.

In some embodiments, an agent that has a tissue type-specific labeling effect has been applied to the area of interest prior to operation of the instrumented biopsy probe. For example, the agent may be a fluorescent marker or Raman spectroscopic marker that labels cells of a particular type for detection via optical means, or a molecularly targeted imaging contrast agent such as a molecularly functionalized microbubble that can be detected acoustically. The environment sensors 122 of the needle can then detect the presence of the labeling agent in the vicinity of the needle. The acquired sample sensors 102 and the controller 140 can then confirm acquisition of the suspect tissue by detection of the labeling agent within the acquired sample by the sample sensors 102 and operation of the sample comparison module 144 as described further below.

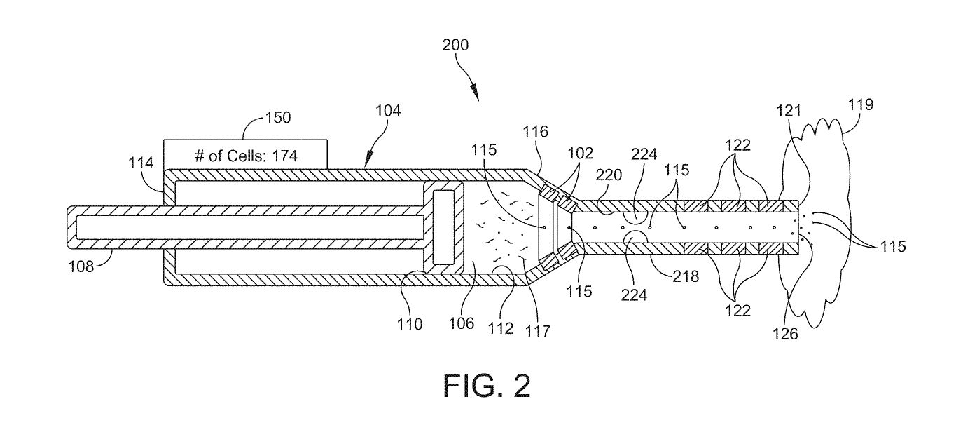

FIG. 2 shows another embodiment of an instrumented biopsy probe 200 that is identical to the instrumented biopsy probe 100 of FIG. 1, except the needle 218 also includes a region of reduced diameter defined by protrusions 224 formed on the inner surface of the lumen 220 of the needle 218. This area of reduced diameter helps to center the cells within the lumen. In some embodiments, this region of reduced diameter helps to create a single file stream of cells passing through the lumen 120 distally of the protrusions 224.

FIG. 3 shows the controller 140 may include a sample evaluation module 142. The sample evaluation module 142 compares the properties of the acquired samples to a database of known properties of normal and abnormal cells to provide guidance to the surgeon as to whether suspicious cells and/or abnormal cells have been captured. For instance, the sample evaluation module 142 is capable of detecting cell size that is abnormal. An abnormal cell size is a property that often requires further analysis by a surgeon. The sample evaluation module 142 is capable of evaluating other properties of interest, including cell wall thickness, overall electrical impedance properties, fluorescence-based light emission, IR signature, mechanical properties such as elasticity, and other properties.

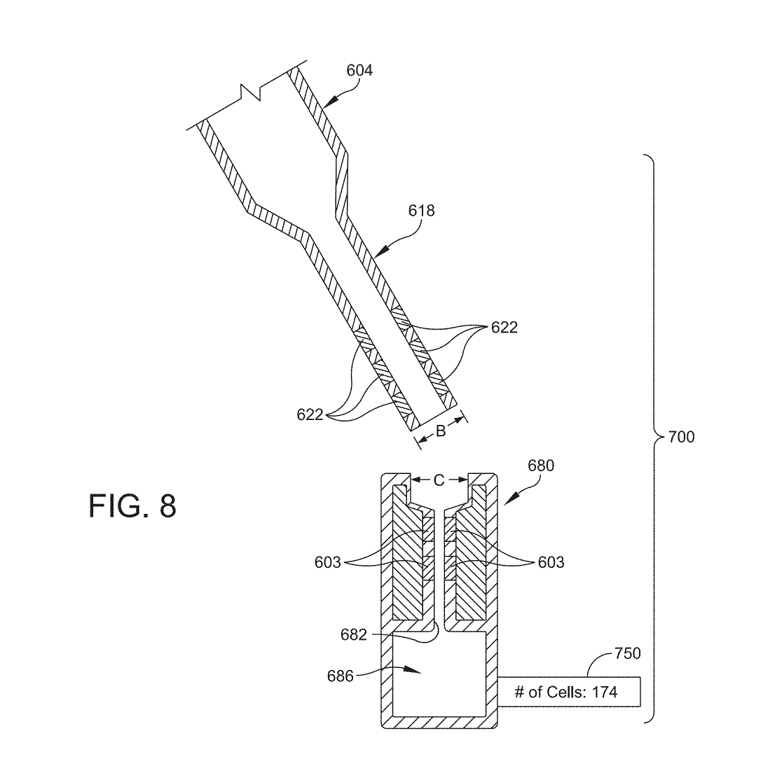

As discussed in relation to the method of FIG. 9, the controller 140 of FIG. 3 may include an automatic trigger that causes the medical instrument to initiate sample collection when the at least one environment sensor 122 detects a suspicious tissue. The automatic trigger can be implemented as hardware or software. In some embodiments, a user may override the automatic trigger. In some embodiments, the automatic trigger causes the medical instrument to initiate sample collection when the environment sensor(s) 122 output measured values that fall within a known range of values corresponding to an abnormal condition, such as the range discussed in relation to the sample evaluation module 142 herein.

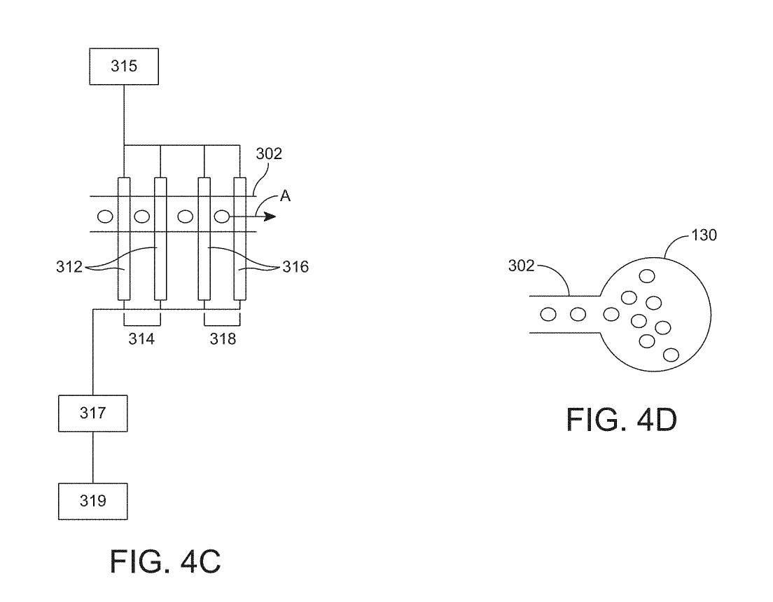

A sample comparison module 144 may compare acquired cells to cells in the surrounding environment. The sample comparison module 144 may compare at least one measured value relating to the acquired sample to at least one measured environmental value of the tissue from which the sample is acquired. The controller 140 is in communication with a user interface, such as a display 150 or another indicator that is configured to provide a notification to a user of the medical instrument based on at least one property of the sample. The sample comparison module 144 communicates with the display 150 to provide the surgeon with an indication on the display 150 of whether the cells of interest were captured in FIGS. 1 and 2. Similarly, FIG. 5 shows an indication 452 on the display 450 of the number of cells of interest that were captured.

Similarly, the sample evaluation module 142 may communicate with the display 150 to cause the display 150 to indicate whether the acquired sample has properties that match properties of known tissues and cells, such as cancer tissues and cells. In some embodiments, the sample evaluation module 142 is configured to compare at least one measured value from the acquired sample sensor 102 to at least one a known range of values corresponding to a normal condition and/or a known range of values corresponding to an abnormal condition.

The controller 140 comprises a memory component and a processor connected to the memory component. The memory component and the processor are connected to the sample evaluation module 142 and to the sample comparison module 144. The controller 140 may be preprogrammed, for example, by computer instructions stored on a computer readable medium or device, such as a hard disk drive, an optical disk readable by an optical disk reader, a flash memory device, and the like. In some embodiments, the controller 140 may be connected to an external power supply. In some embodiments, the controller may include an internal power supply. The controller includes system interface components to send or receive data with other components, such as the acquired sample sensors 102, the environment sensors 122, and the display 150. The system interface components may include hardware components, software components, or a combination of hardware and software components. The controller 140 may be incorporated on an application-specific integrated circuit (ASIC). Components of the controller such as the sample evaluation module 142, the sample comparison module 144, the data collection module 146 and the navigation module 148 may include hardware components, software components, or a combination of hardware and software components.

In some embodiments, the sample comparison module includes a memory component and a processor connected to the memory component.

In some embodiments, the sample evaluation module includes a memory component and a processor connected to the memory component.

The display 150 may include lights, text, or other visual alerts. Additionally, the controller 140 can be connected to an audible output device that provides audible alerts to a surgeon.

While FIG. 3 shows the controller 140 including a sample evaluation module a sample comparison module, a data collection module and a navigation module, it is understood that in some embodiments, the controller may include only some of these modules and/or utilize only of these modules.

In some embodiments, the controller 140 may be incorporated into the medical instrument, such as the biopsy probe 100. In some embodiments, a system for collecting a sample may include the biopsy probe and a peripheral device, and the controller 140 may be incorporated into the peripheral device. The peripheral device may be a computation/database unit. The peripheral device may be in communication, either wired or wirelessly, with the biopsy probe. The computation/database unit may perform data analysis and may provide feedback to the surgeon. The feedback to the surgeon, or other healthcare provider or user of the system for collecting a sample, may include a notification.

In response to a notification on the display 150, a surgeon or other user of the device may adjust the sample acquisition technique, such as by adjusting vacuum strength, using a chemical disrupting agent, activating an ultrasonication mechanism, or by using another method that is appropriate for the sample acquisition technique. For example, the user of the device may adjust the sample acquisition technique in response to the number of cells or properties of cells that have been acquired. Such adjustments may be performed to improve the quality and/or quantity of the acquired samples.

The controller 140 may also include a data collection module 146 that stores the physical properties of both the acquired sample and/or the environment that the sample was acquired from in a database for eventual correlation to pathology results. This enables ongoing usage of the biopsy probe to generate a database of tumor properties that may be employed by the database unit to provide real-time feedback to the surgeon.

The controller 140 may also include a navigation module 148, linked to medical imaging, that captures the location at which the sample is acquired, as well as any physical properties of that location such as the pH, elasticity, and so forth of the tissue at the point it was acquired. In this way, sensor data generated by the on-board analysis of the probe may be tied together or “stamped” with sample acquisition time, sample acquisition location, and sample capture conditions (for example, vacuum pressure or temperature).

In FIG. 1, the display 150 is mounted on the instrument body 104 of the biopsy probe 100.

In some embodiments, the user interface, such as display 150, may be on a peripheral device. In some embodiments, a user interface may be on a body of a device, such as on the handle of a biopsy needle.

Counting how many cells or clusters of cells are present facilitates improving the effectiveness of biopsy procedures by providing surgeons with timely and quantitative feedback during the biopsy procedure. An exemplary embodiment of this disclosure involves use of a cell counter to count the acquired cells (or clusters of cells) in real-time as they enter the sample collection instrument. This counting may be achieved electrically using impedance spectroscopy, for instance using a device such as the Coulter Counter microfluidic device schematically illustrated in FIG. 4C.

FIGS. 4A-4D show components that may be incorporated in a medical instrument according to the present disclosure.

A medical instrument according to the present disclosure may include a sample preparation device. As noted above, FIG. 4A illustrates an end view of a filter 126 that is positioned at the proximal end 121 of the needle 118. Pores 128 are defined in the filter 126 to allow single cells to pass through the filter 126 and ensure that single cells, rather than aggregates of cells, are being detected by the cell counter of the instrumented biopsy probe.

The filter 126 of FIG. 4A is useful in various medical instruments, such as a biopsy needle, that are used in medical procedures where aggregates of cells may be encountered. Aggregates of cells may be found in living tissue, such as living human tissue, or may be found in ex vivo tissue.

Other sample preparation modules can be incorporated into the biopsy device to render the sample more amenable to on-board analysis. Such devices may include a filtration module to eliminate via size-exclusion tissues that are of interest from tissues that are not of interest, a dilution module to adjust the concentration and/or viscosity and/or buoyancy of the sample to render it amenable to sampling by techniques such as electrical impedance characterization, a mixing module, an ultrasonication module that breaks-up clusters of cells, and a hydrodynamic focusing module that steers the cells towards the center of the flow channel by partially or completely surrounding them with a guiding buffer solution.

FIG. 4B illustrates an embodiment of a flow-focusing device. In some embodiments, the flow-focusing device 300 of FIG. 4B is included in a cell counter microfluidic chip 309 fabricated using a material that is suitable for fabricating a chip 309 and that is biocompatible. In some embodiments, the chip 309 is made of a material such as an elastomer, a thermoplastic, an epoxy, or a cyclic olefin copolymer. In some embodiments, the chip 309 is made of a material typically used to mold a syringe. In some embodiments, the chip 309 is injection molded. In some embodiments, the chip 309 is 3D-printed.

In one example, the cell counter microfluidic chip 309 is fabricated using a metallized glass substrate bonded to a polydimethylsiloxane (PDMS) molded channel. In the flow-focusing device, center microfluidic channel 302 extends from a first end 304 to a second end 306. Two side inlets 308 may provide buffer solution for hydrodynamic focusing of the flow, while the solution 310 containing cells to be counted flows through the center microfluidic channel 302. As a fluid containing cells flows from the first end 304 to the second end 306, a second fluid flows from the side inlets 308 into the center microfluidic channel 302 to focus the flow.

In some embodiments, the flow-focusing device may focus flow in two dimensions. In some embodiments, the flow-focusing device may focus flow in three dimensions.

In some embodiments, the flow-focusing device 300 of FIG. 4B may be incorporated on a structure other than a chip.

Downstream from the flow-focusing device in FIG. 4B is a detection region in FIG. 4C having a set of first electrodes 312 mounted adjacent the center microfluidic channel 302. The set of first electrodes 312 detect cells as they flow in the direction of arrow A through a first zone of 314 in the channel 302 via electrical impedance measurement. A set of second electrodes 316 mounted on the sides of the center microfluidic channel 302 detect cells as they flow through a second zone 318 of the channel 302 via electrical impedance measurement. A function generator 315 is connected to the electrodes, a transimpedance amplifier 317 and a data acquisition card 319 is connected to the electrodes to measure impedance.

In some embodiments of medical instruments according to the present disclosure, acquired sample sensors, such as acquired sample sensors 102 of FIG. 1 may include the function generator 315, transimpedance amplifier 317, and the data acquisition card 319.

In some embodiments, the sample sensors 102 are formed as the set of first electrodes 312 and the set of second electrodes 316.

In some embodiments, additional electrodes may be included.

A plurality of flow-focusing devices and detection regions such as the structures of FIGS. 4B and 4C may be utilized in parallel to provide sufficient flow rate to process the samples in real-time as they flow through the needle.

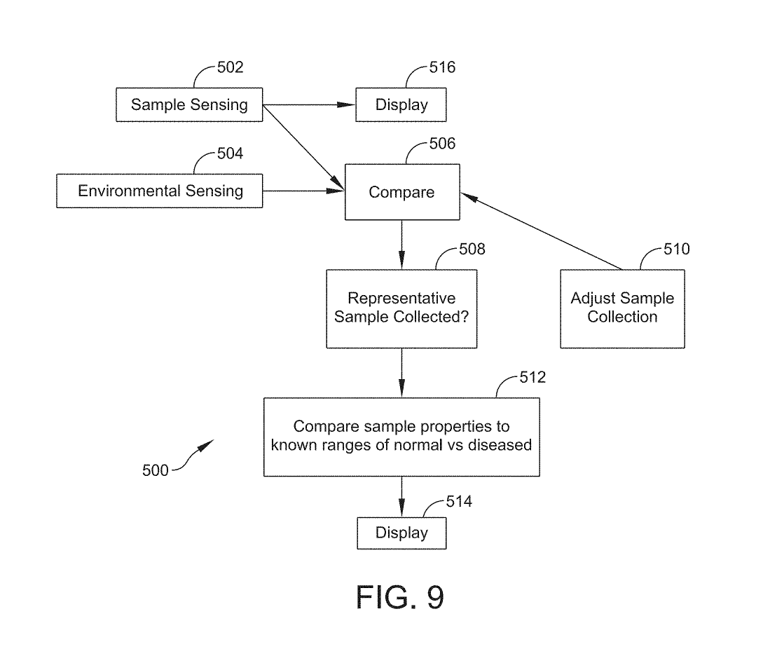

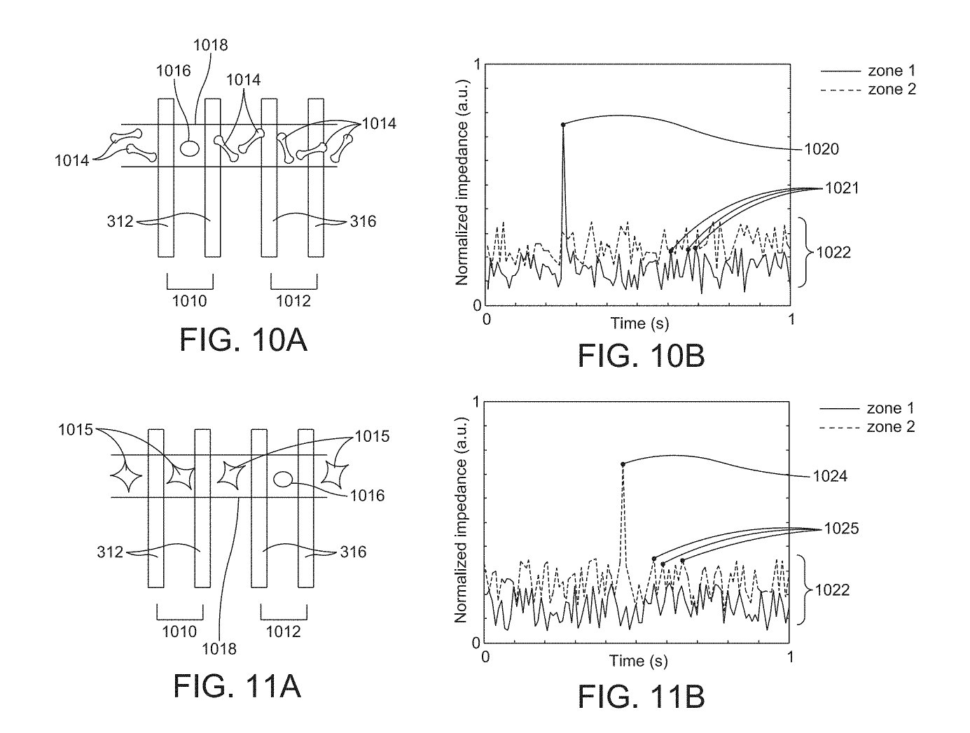

Counting of cells or clusters of cells may also be achieved using other methods, such as optical detection, acoustic detection, or measurement of other physical, chemical, or mechanical properties of the cells. Examples of cell counting using a prototype electrical counter chip are shown in FIGS. 10A and 11A.

In some embodiments, a detection region, such as the detection region of FIG. 4C, may be provided without the flow-focusing device of FIG. 4B. In some embodiments, a detection region, such as the detection region of FIG. 4C, may be provided without the filter of FIG. 4A. In some embodiments, a detection region, such as the detection region of FIG. 4C, may be provided for real-time sensing of a sample in a device without the flow-focusing device of FIG. 4B, without the filter of FIG. 4A, and without the collection chamber of FIG. 4D.

FIG. 4D shows a collection chamber 130 for collecting the sample after the sample has passed through the detection region. The collection chamber 130 can be included in a hand-held device, such as a biopsy probe, or can be included in a peripheral device, such as an ultrasound machine or a base unit. The collection chamber 130 can be connected to the center microfluidic channel 302 of FIG. 4C, downstream of the detection region of FIG. 4C.

FIG. 5 shows a cross-sectional view of an embodiment of an instrumented biopsy probe 400 with internal acquired sample sensors 402 to detect, quantify, and characterize acquired samples and external sensors to characterize the tissue. The instrumented biopsy probe 400 includes a sample preparation module 440 that performs functions such as mixing, dilution, and hydrodynamic focusing of a solution.

The instrumented biopsy probe 400 includes a syringe body 404 having a cylindrical body portion 406. A plunger 408 is positioned within the cylindrical body portion 406. An outer edge 410 of the plunger engages an inner surface 412 of the cylindrical body portion 406. The plunger 408 is movable within the cylindrical body portion 406. Movement of the plunger towards the distal end 414 of the cylindrical body portion 406 creates negative pressure within the cylindrical body portion 406.

A frustoconical portion 416 extends from the cylindrical body portion 406. A needle 418 having a lumen 420 defined within the needle 418 extends from the frustoconical portion 416 at the proximal end 421 of the syringe body.

A user can operate the biopsy probe 400 to draw fluid and cells into the syringe body 404. Moving the plunger 408 distally within the cylindrical body 406 creates negative pressure within the syringe body 404 to draw a sample 417 into the biopsy needle from a surrounding environment, such as tumor 419.

FIG. 5 shows the proximal end of the needle 418 positioned adjacent to a tumor 419. The sample may include fluid and cells. If the region of interest is truly a tumor 419, and if the biopsy is successful, the sample includes cells from the tumor.

The proximal end of the needle 418 may include a filter 426, such as the filter shown in FIG. 4A.

To determine whether the sample includes cells from the tumor 419, at least one acquired sample sensor 402 is secured to the inner surface of the frustoconical portion 416. FIG. 5 shows two sample sensors 402 secured to the inner surface of the frustoconical portion 416. In some embodiments, the acquired sample sensors 402 are secured to the inner surface of the frustoconical portion 416 using an adhesive. In some embodiments, the acquired sample sensors 402 are embedded in the inner surface of the frustoconical portion 416.

To determine whether the proximal end of the needle 418 is located adjacent a suspicious tissue, the proximal end of the needle 418 includes at least one environment sensor 422. FIG. 5 shows six environment sensors 422 secured to an outer surface of the needle.

A sample preparation module 440 may be located along the length of the needle 418. The sample preparation module 440 includes a hydrodynamic focusing element that centers the flow of the acquired cells in the portion of the lumen 442 that is located in the lumen of the needle in the distal end of the sample preparation module 440. This flow-focusing may occur in either two dimensions or in three dimensions. In FIG. 5, the sample preparation module 440 includes two channels 444 that each intersect with the needle 418 at an angle to focus the flow within the lumen 442.

The instrumented biopsy probe 400 of FIG. 5 includes a user-interface display 450 that indicates in real-time the number of cells that have been acquired. The user interface provides feedback, such as indication 452, to the surgeon about the physical properties of the acquired samples during the biopsy procedure. In FIG. 5, the user interface display 450 shows the indication 452 “# of cells: 174.” This indicates to a surgeon, or another user of the biopsy probe 400, that 174 cells were counted. In some embodiments, the indication 452 relates to a number of cancer cells counted, a number of suspicious cells counted, or a number of abnormal cells counted.

The user interface display updates the information as cells are collected and counted.

In some embodiments, the biopsy device is capable of performing methods such as electro-cauterization, electro-ablation, or chemical encapsulation may be employed to reduce chances of biopsy-induced metastasis induced by the disturbance introduced by the biopsy needle (so-called “needle-tract metastases”). The present disclosure introduces the practice of triggering (or regulating) the use of these anti-metastasis techniques selectively based on detection of a sufficient collected sample. In other words, encapsulation or electro-ablation is delayed until successful sample acquisition has been confirmed by real-time sensing, which is on-board sensing in some embodiments.

In some embodiments, the biopsy device has a modular design in which a disposable “needle” component may be secured to a reusable “holder,” which contains reusable elements such as the user interface/display, power supply, light sources, pumping and/or vacuum pressure sources, optical detectors, and communication modules. The disposable component contains the parts that actually contact the patient samples, such as the needle itself, sensors that require physical contact with the sample, and sample preparation devices.

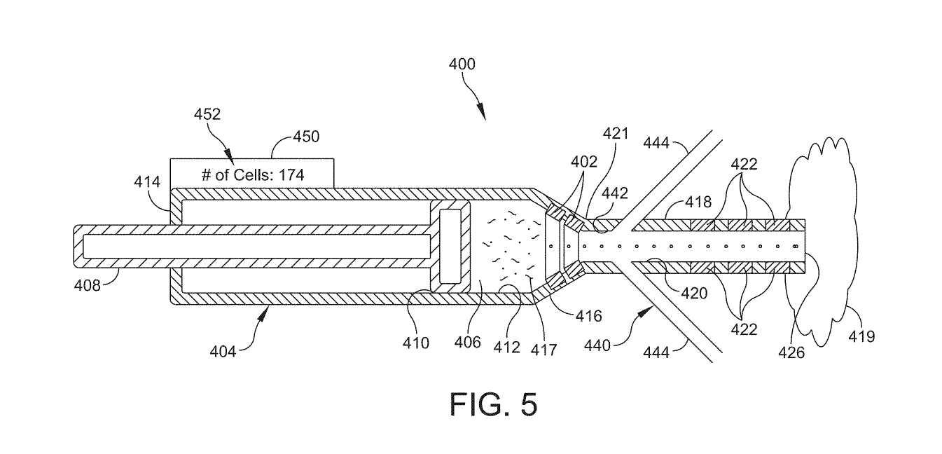

For example, FIG. 6 shows another embodiment of a medical instrument according to the present disclosure, in which the medical instrument has removable parts.

The instrumented biopsy probe 600 may include an instrument body 604 having a cylindrical body portion 606. A plunger 608 may be positioned within the cylindrical body portion 606. An outer edge 610 of the plunger engages an inner surface 612 of the cylindrical body portion 606. The plunger 608 may be movable within the cylindrical body portion 606. Movement of the plunger towards the distal end 614 of the cylindrical body portion 606 creates negative pressure within the cylindrical body portion 606. In some embodiments, a vacuum source such as a pump is used in place of the syringe to provide negative pressure in the instrumented biopsy probe.

In some embodiments, a frustoconical portion 616 extends from the cylindrical body portion 606. A needle 618 having a lumen 620 defined within the needle 618 extends from the frustoconical portion 616 at the proximal end of the syringe body.

The needle 618 is removable from the syringe body 604. In some embodiments, the needle 618 includes external sensors 622 to characterize the tissue surrounding the needle 618 at the external sensors 622.

A sensing cartridge 670 is shown seated within the syringe body in FIG. 6, but a user can remove the sensing cartridge from the syringe body. The cartridge 670 includes internal acquired sample sensors 602 to detect, quantify, and characterize acquired samples.

The cartridge 670 is oriented within the syringe body 604 so that the sample passes from the lumen of the needle 618 to a channel 672 defined in the cartridge 670. The acquired sample sensors 602 are positioned along the channel 672. The distal portion of the cartridge 670 includes an annular wall 674 that is dimensioned to sealingly engage an inner surface 612 of the cylindrical body portion 606 the syringe body 604. Together, the annular wall 674 of the cartridge 670, the inner surface 612 of the cylindrical body portion 606 of the syringe body 604 and the plunger 608 define a sample collection region 676. The sample that passes through the needle 618 cannot enter the sample collection region 676 without passing through the channel 672.

A user may operate the plunger 608 of the instrument body 604 to draw fluid and cells into the instrument body 604. Moving the plunger 608 distally toward the distal end 614 of the cylindrical body portion 606 within the cylindrical body 606 creates negative pressure within the instrument body 604 to draw a sample into the biopsy needle from a surrounding environment.

The proximal end 621 of the needle 618 includes a filter 626, which can be constructed as the filter shown in FIG. 4A. The filter 626 includes pores that are dimensioned to prevent large aggregates of cells from entering the needle. In some embodiments, the filter 626 includes pores that are just large enough to allow certain cells, such as cancer cells and blood cells, to pass through the pores of the filter 626 and into the needle 618.

In some embodiments, the proximal end 621 of the needle 618 is actuated. For example, an actuator can be used to oscillate the proximal end 621 of the needle 618. This oscillation is useful for breaking up aggregates of cells into individual cells that can pass into the needle through the filter 126.

The sample sensors 602 and the environment sensors 622 are connected to the controller 140 either through a wired or wireless connection. The sample sensors 602 and controller 140 can be configured to operate to characterize the acquired sample in the same ways as the sample sensors 102 in relation to FIG. 1 and controller 140. The environment sensors 622 and controller 140 can be configured to operate in the same ways as the environment sensors 122 in relation to FIG. 1 and controller 140.

FIG. 6 shows a display 150 secured to the syringe body 604.

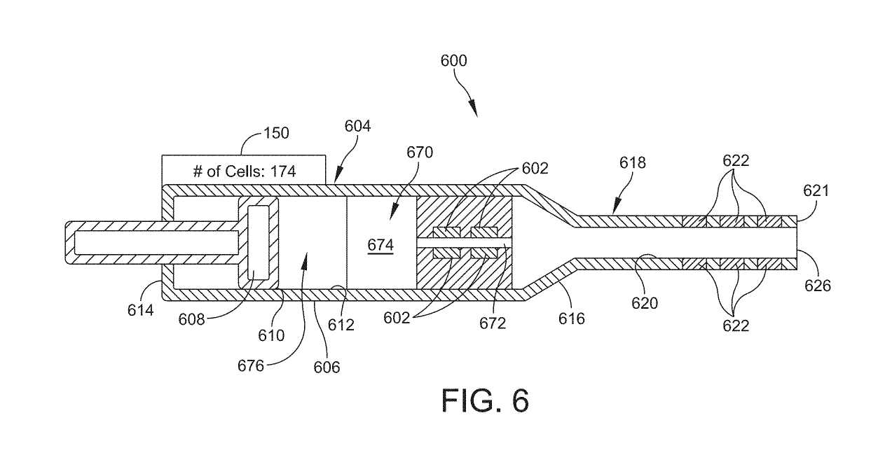

In some embodiments, the sample sensors are not included in the biopsy device, but are instead included in a reservoir on a peripheral device. FIG. 7 shows an assembly 700 that includes the syringe body 604 and needle 618 of FIG. 6, but not the cartridge 670. In place of the cartridge 670, a peripheral device 680 is provided. The peripheral device 680 includes a recess defined by an annular wall 681. The recess is dimensioned to receive the proximal end 621 of the needle 618.

In some embodiments, the peripheral device 680 may be configured as a sleeve that extends over a greater length of the needle 618. For example, the annular wall 681 has a height that is closer to the length of the needle 618.

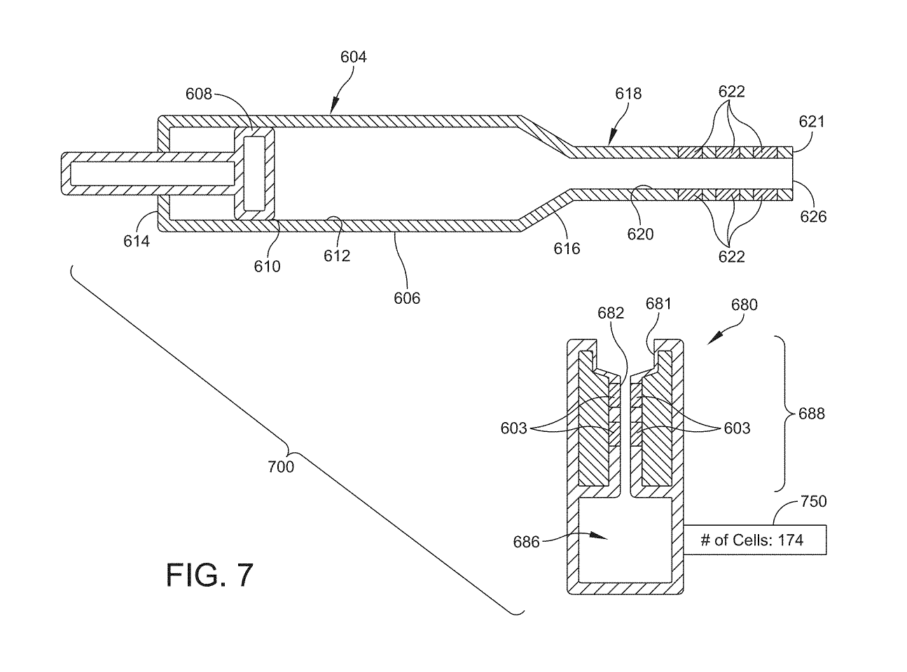

FIG. 8 shows that the needle 618 has an outer diameter B and the annular wall 681 of the peripheral device 680 has an inner diameter C. C is at least as great as B. In some embodiments, B may be dimensioned to provide a sealing engagement between the outer surface of the needle 618 and the annular wall 681. In some embodiments, the recess may be sufficiently large that it provides a well into which the user can dispense the entire sample collected.

The peripheral device 680 has a sample detection region 688 and a sample collection region 686. The sample detection region 688 of the peripheral device 680 includes internal acquired sample sensors 603 to detect, quantify, and characterize acquired samples as the sample passes from the recess to the collection region 686.

The cells of interest, such as cancer cells and blood cells, in the sample cannot enter the sample collection region 686 without passing through the channel 682.

The peripheral device includes a display 750, which can be configured similarly to display 150, as described in relation to other embodiments. For example, display 750 can be connected to controller 140.

In some embodiments, the peripheral device can include a controller, such as controller 140.

Acquired sample sensors 603 are similar in operation to the acquired sample sensors 602, and are connected either wirelessly or through a wired connection to the controller 140.

The dimensions of the peripheral device may be altered without departing from the scope of the present invention. For example, a user may wish to enlarge the sample collection region 686 depending on the amount of sample being collected. Similarly, the diameter of the channel 682 may be altered depending on the sample being acquired and depending on the size of the cells that the user is counting.

The peripheral device may be an ultrasound machine or another device.

FIG. 9 shows a block diagram of an embodiment of a method 500 of collecting a sample. The method can be performed using, for example, the biopsy probe 100 and controller 140 or other embodiments according to the present disclosure. A user maneuvers a medical instrument in a sample environment, such as a tissue. The sample is collected from a sample environment using a device, such as any device of the present disclosure, for example biopsy probe 100 of FIG. 1. At 502, sample sensors identify at least one property of the sample and provide data relating to the one or more properties of the sample to a sample comparison module. At 504, environment sensors identify at least one property of the sample environment (for example, tissue) and provide data relating to the one or more properties of the environment to the sample comparison module. At 516, the display shows information relating to the acquired sample. At 504, in some embodiments, the controller determines if the environment sensors on the needle have detected a suspicious tissue, thereby determining whether the needle is in the presence of a suspicious tissue. In some embodiments, if the controller determines that the needle is in the presence of a suspicious tissue, the controller triggers sample collection from that suspicious tissue.

At 506, the comparison module collects the data from the acquired sample sensors and the environment sensors and sends it to the data collection module.

At 508, the comparison module determines whether properties of the acquired sample match those of the tissue. This determination can be performed on either the medical instrument or a peripheral device. If the properties of the acquired sample do not match those of the sample environment tissue, a feedback loop leads to 510 and then back to 506. In some embodiments, at 510, the controller 140 instructs an actuator 155 of the device (or outputs an instruction to the user of the device through a visual or audible notification), in real-time while the medical instrument (medical device) is being maneuvered, to adjust the sample collection technique, for example to increase acquisition technique aggressivity or to change the position or orientation of the medical instrument. In some embodiments, the controller outputs an instruction in real-time during the step of maneuvering the device. In some embodiments, the controller outputs an instruction in real-time after the user has removed the medical instrument from the patient, but before the procedure has concluded.

The increase in acquisition technique aggressivity can include an adjustment of vacuum pressure, ultrasonication, chemical disrupting agent, agitation, other parameters, or any combination of these to encourage the sample to be acquired. At 506, the sample comparison module, such as sample comparison module 144, collects the data of the new sample, which leads to 508.

When properties of the acquired sample match those of the tissue at 508, at 512 sample properties are evaluated relative to a database of known properties of normal vs. diseased tissues to determine whether the collected sample is suspicious for being diseased. This can be performed using the sample evaluation module 142.

At 512, the method can also determine whether the collected sample is abnormal.

In some embodiments the sample includes a plurality of cells. In some embodiments, the plurality of cells includes at least one normal cell and at least one abnormal cell. In some embodiments, the at least one normal cell is non-cancerous and the at least one abnormal cell is cancerous. At 512, the method can determine whether a cell is cancerous.

The method includes informing a user of information related to a property of the sample and/or a property of the environment. At 514 results are displayed on a user interface, typically a display, or optionally a tactile feedback mechanism or an audio feedback mechanism to inform the surgeon of the results. The display can be display 150 of FIG. 3.

In some embodiments, the method can also determine whether the collected sample is suspicious for being diseased.

The method 500 can use any of the devices and systems disclosed herein, such as the biopsy probe of FIG. 1, the biopsy probe of FIG. 2, the chip of FIG. 4C, the biopsy probe of FIG. 5, the biopsy probe of FIG. 6, the biopsy probe of FIGS. 7-8, and other embodiments in accordance with the present disclosure.

The method and devices described herein can be incorporated in an ultrasound procedure or an ultrasound device.

### EXAMPLE 1

The present disclosure provides one or more sensors capable of detecting the size of a cell passing over the one or more sensors. In one example, sensors of the present disclosure are useful for detecting cancer cells that are mixed with red blood cells and plasma.

FIG. 10A shows a cell counter with a set of first electrodes 312 in a first zone 1010 and a set of second electrodes 316 in a second zone 1012. As red blood cells 1014 and a cancer cell 1016 mixed in a fluid such as plasma and/or another blood component pass over the electrodes 312, 316, the impedance is measured on the electrodes 312, 316. The normalized impedance for the first zone 1010 is plotted over time in FIG. 10B and is labeled “zone 1.” The normalized impedance for the second zone 1012 is plotted over time in FIG. 10B and is labeled “zone 2.” The peak 1020 in the “zone 1” plot in FIG. 10B shows the time at which the cancer cell 1016 was in the first zone 1010. Additional peaks 1021 in the range 1022 in the “zone 1” plot show times at which red blood cells 1014 were in the first zone 1010. The controller 140 may be operated to count the number of peaks 1020 over a period of time. Because the channel 1018 through which the red blood cells 1014 and cancer cell(s) 1016 are passing is sufficiently narrow, the cells pass through the channel substantially in single-file.

In some embodiments, flow-focusing structures and methods described herein may be used to cause the cells to pass through the channel substantially in single-file. In some embodiments, flow-focusing structures and methods described herein may be used to cause the cells to pass through the channel in single-file. In some embodiments, both the diameter of the channel 1018 and flow focusing methods/structures may together cause the cells to pass through the channel substantially in single-file. In some embodiments, both the diameter of the channel 1018 and flow focusing methods/structures may together cause the cells to pass through the channel in single-file.

Because of this, the number of peaks 1020 in a given period of time is a close approximation or exactly matches the number of cancer cells that pass through the first zone 1010 of the channel 1018 in that given period of time.

### EXAMPLE 2

Similarly, in another example, sensors of the present disclosure are useful for detecting cancer cells that are mixed with normal cells in a fluid, such as a saline buffer, another buffer fluid, or another fluid useful for mixing cells.

FIGS. 11A and 11B show how cancer cells 1016 may be counted and differentiated from normal cells 1015, such as normal human cells. Example 2 uses the same cell counter as in Example 1.

FIG. 11A shows the cell counter of FIG. 10A. The normalized impedance for the first zone 1010 and the normalized impedance for the second zone 1012 are each plotted over time in FIG. 11B. A user can count the number of peaks 1024 in the “zone 2” plot of FIG. 11B. The number of peaks 1024 corresponds to the number of cancer cells 1016 passing through the second zone 1012 of the channel 1018 in that given period of time. The number of peaks 1025 in the range 1022 corresponds to the number of normal cells 1015 passing through the second zone 1012 of the channel 1018 in that given period of time.

A large body of literature exists on technologies to steer biopsy needles, to determine the location of a biopsy needle, and to characterize the microenvironment that the needle is in. For instance, work on Electrical Impedance Spectroscopy seeks to characterize the needle environment, while other work utilizes acoustic properties to characterize the needle's environment. Others have used an optical approach, an electrical approach, and/or an acoustic approach to characterize the properties of the needle's environment for tissue types such as prostate and breast. These existing approaches may be used in combination with the present disclosure, to help to determine whether the needle is in the presence of a suspicious tissue, so as to trigger sample collection at the proper moment in the proper location. Others propose a method for sensing the shape or position of a needle and for controlling that shape in a manner that is compatible with magnetic resonance imaging of the needle's position.

These existing approaches are geared towards steering the needle towards the tissue of interest, either via medical imaging (for instance ultrasound-guided biopsy) or via direct sensing of the microenvironment, and towards detecting when the proper location has been reached. However, even though the sample collection may have been triggered in the proper location, the sample collection may not have been successful in acquiring the targeted cells. Existing technologies do not characterize the acquired sample itself, so do not provide feedback as to whether sample collection is adequate to enable instant analysis via on-board sensing, or later pathological analysis in a lab.

The present disclosure provides an approach that is unique in that it incorporates sensors that characterize the acquired sample and provide real-time feedback to the surgeon. Feedback may be a simple characterization of a physical or chemical property such as pH or number of cells, or may be a more complex analysis that compares the sample properties to those in the environment of the needle (for instance how much tissue was acquired that has the same green tint that is seen on the exterior of the needle), or via comparison to a database of known properties of suspicious tissue types (how many very-thin-walled cells were acquired, or how many elongated cells were acquired). Sample properties detected by the devices and methods of the present disclosure may also include a number of red blood cells collected in a sample, a number of cells collected that are not red blood cells, a ratio of the number of red blood cells in a sample to the number of cells in the sample that are not red blood cells, a number of cancer cells collected in a sample, types of cancer cells collected in a sample and any combination of these.

The present disclosure provides an approach that provides feedback that permits the surgeon to know in real-time whether the desired samples were acquired. Knowing this permits real-time alteration of sample acquisition properties, such as use of increase vacuum force or increase agitation or ultrasonication. In general one wants to be as gentle as possible, minimizing disruption of the tissue so as to reduce the risk of inducing metastases. However, if one is too gentle, the necessary cells are not acquired. Having feedback from on-needle sensors will for the first time provide surgeons with measurable data with which to decide how much tissue disruption is needed to acquire a sample effectively. The degree of disruption may be adjusted manually via the surgeon modifying their surgical technique, or may be achieved automatically by the probe itself adjusting acquisition properties such as vacuum pressure so as to optimize capture of needed material while minimizing tissue disruption. Additionally, for difficult to acquire tumor sites, such as metastasized tumors, which may be difficult to locate and sample, knowing in real-time that a suspicious cell was acquired may be of great value in terms of knowing how many samples to acquire, what volume of sample to acquire, and under what tissue-disruption conditions to acquire those samples.

The present disclosure could be applied to a medical instrument, such as biopsy needle (for example, as shown in relation to FIG. 1 and FIG. 5), a biopsy punch, an excision tool, or another medical instrument.

The sensors may be applied in or on surfaces of a medical instrument, or may be embedded within a body of a medical instrument.

As used herein, suspicious tissue refers to tissue that is suspicious of being diseased, infected, or otherwise indicating an abnormal condition of interest.

Embodiments are not limited in their application to the details of construction and the arrangement of components set forth in the following description or illustrated in the drawings. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having thus described several aspects of at least one embodiment, it is to be appreciated various alterations, modifications, and improvements will readily occur to those skilled in the art. Such alterations, modifications, and improvements are intended to be part of this disclosure, and are intended to be within the scope of the disclosure. Accordingly, the foregoing description and drawings are by way of example only.

What is claimed is:

1. A method of guiding sample collection, the method comprising:
maneuvering a device in a tissue;
collecting a sample from the tissue using the device;
identifying a property of the sample;
identifying a property of the tissue;
determining whether the property of the sample matches the property of the tissue; and
outputting a real-time instruction during the step of maneuvering the device to increase sample acquisition aggressivity if the property of the sample does not match the property of the tissue.

2. The method of claim 1, wherein the step of outputting the instruction comprises one of outputting the instruction to a user and outputting the instruction to a controller.

3. The method of claim 1, wherein increasing sample acquisition aggressivity includes at least one of adjusting of vacuum pressure, adjusting ultrasonication, adding a chemical disrupting agent, and agitating.

4. The method of claim 1, wherein the step of determining is performed on one of the device and a peripheral device.

5. The method of claim 1, further comprising determining whether the collected sample is abnormal.

6. The method of claim 1, further comprising informing a user of information related to at least one of the property of the sample and the property of the environment.

7. The method of claim 1, wherein the sample includes a plurality of cells.

8. The method of claim 7, wherein the plurality of cells includes at least one normal cell and at least one abnormal cell.

9. The system of claim 8, wherein the at least one normal cell is non-cancerous and the at least one abnormal cell is cancerous.
